(19) United States
(12) Patent Application Publication
(10) Pub. No.: US 2003/0083652 A1
(43) Pub. Date: May 1, 2003

(54) Method for treating tissue in arthroscopic environment

(75) Inventor: Mark D. Markel, Madison, WI (US)

Correspondence Address: JOEL R. PETROW, SMITH & NEPHEW, INC., 1450 BROOKS ROAD, MEMPHIS, TN, 38116, US

(73) Assignee: ORATEC INTERVENTIONS, INC

(21) Appl. No.: 09/996,978

(22) Filed: Oct. 31, 2001

(51) Int. Cl. A61B 18/14

(52) U.S. Cl. 606/41

(57) ABSTRACT

A method for treating tissue having a surface in an arthroscopic environment of a mammalian body having a body temperature with a probe having a proximal end and an electrode at a distal end. The method includes the steps of providing a warmed irrigating solution having a temperature approximating the body temperature, delivering the warmed irrigating solution into the arthroscopic environment, introducing the distal extremity of the probe into the arthroscopic environment, positioning the electrode adjacent the surface of the tissue and supplying thermal energy to the electrode so as to treat the tissue. The warmed irrigating solution inhibits undesirable heating below the surface of the tissue.

### BACKGROUND OF THE INVENTION

1. Field of the Invention

This invention relates to a new and improved method for treating tissue in an arthroscopic environment of a mammalian body.

2. Description of Related Art

The normal function of joints in humans depends on the distribution of relatively large forces across the body surfaces. In diarthrodial joints, the magnitude of the joint forces reaches levels four to seven times body weight. These forces are dispersed by articular cartilage in the joint. Proper cartilage function occurs via a highly organized extracellular matrix maintaining a fixed charge density and possessing a high affinity for water.

Chondromalacia occurs when cartilage beds in joints become worn and degenerate into strands of cartilage which extend away from their respective cartilage beds and into the joint cavity. The cartilage surface becomes visibly disrupted, fissured and fibrillated. The damaged cartilage has deleterious effects on the mechanical properties and normal function of articular surface. The fibrillated cartilage may breakdown and break off to form particulate matter. It is the particulate matter (broken fibrils) and various proteins and enzymes released when the normally smooth layered architecture of cartilage is undermined and frayed, which causes pain by irritating the synovial lining of the joint.

Treatment to date has included surgical intervention. In one arthroscopic procedure, a shaver is introduced through an arthroscope and is used to mechanically remove the strands of disrupted and fibrillated cartilage. However, this treatment can disrupt and remove part of the normal healthy cartilage bed and does not restore a smooth surface nor improve the mechanical function. In fact, mechanical shaving has several drawbacks including: 1) adjacent normal cartilage is often removed while debriding focal lesions; 2) it is difficult to completely smooth the cartilage surface and not leave fine fibrillated regions; and 3) it is a challenge to create a completely smooth cartilage surface with mechanical shaving. After treatment, normal loading typically causes continued degradation that results in further fibrillation and degradation.

By way of example, the thickness of articular cartilage in the region of the femoral condyles is approximately 2-4 mm in humans. Traditional mechanical debridement with shaving systems usually removes 200-400 μm of cartilage including diseased cartilage and underlying normal cartilage if the shaver is well controlled during the treatment. Following mechanical debridement, further chondrocyte death between 100-400 μm deep to the surface occurs within the first two weeks of surgery. Therefore, mechanical debridement with a shaver results in 300-800 μm of chondrocyte loss, due to tissue removal and subsequent chondrocyte death with the cartilaginous surface still microscopically rough following treatment.

Another modality for the repair and treatment of the damaged cartilage includes open procedures which can lead to increased recovery time and a possible increase in pain and further dysfunction of the joint.

The use of thermal chondroplasty for treating cartilage joint surfaces is known and thermal chondroplasty with radiofrequency energy (RFE) has gained widespread use over the past several years. Studies have shown that RFE treatment results in smoother cartilage surfaces than conventional mechanical debridement. Currently, there are two basic RFE systems available for clinical application, monopolar RFE (mRFE) and bipolar RFE (bRFE) systems. In addition, temperature controlled RFE probes and generators are available for clinical application with both monopolar and bipolar RFE systems.

An exemplary device for treating fibrillated cartilage joint surfaces or irregular cartilage joint surfaces in an arthroscopic procedure which delivers sufficient RFE to reduce the level of fibrillation of the cartilage joint surface is described in U.S. Pat. No. 6,068,628 to Fanton et al. Particular care is used to minimize any undesired thermal effect on non-targeted tissue and thereby prevent necrosis below the surface of the cartilage joint surface into the healthy layer since cartilage does not grow and regenerate after being damaged.

Generally, when an arthroscopic procedure utilizes thermal energy, lavage is often used in order to distend the joint cavity and to flush and debris from the joint cavity which is generated during the procedure. Generally, room-temperature lavage is used, however, a trend to use cooled lavage has recently developed. Although using room-temperature and/or cooled lavage is acceptable and generally beneficial for some procedures, during other procedures such lavage may have an undesired cooling effect. In particular, when using a temperature controlled probe having a feedback controller, the feedback controller may cause the probe to overcompensate and actually deliver more energy than is necessary, resulting in deleterious effects on chondrocyte viability.

In view of the foregoing, it would be desirable to provide a method for treating tissue in an arthroscopic environment, for example, to coagulate fibrillated cartilage strands together, without undesirable cooling within the arthroscopic environment which may, in some cases, cause significant chondrocyte death during RFE treatment for thermal chondroplasty.

### SUMMARY OF THE INVENTION

In summary, one aspect of the present invention is directed to a method for treating tissue having a surface in an arthroscopic environment of a mammalian body having a body temperature with a probe having a proximal end and an electrode at a distal end. The method includes the steps of providing a warmed irrigating solution having a temperature approximating the body temperature, delivering the warmed irrigating solution into the arthroscopic environment, introducing the distal extremity of the probe into the arthroscopic environment, positioning the electrode adjacent the surface of the tissue and supplying thermal energy to the electrode so as to treat the tissue. The warmed irrigating solution inhibits undesirable heating below the surface of the tissue.

In general, one advantage of the present invention is to provide a method for delivering energy within a arthroscopic environment to a targeted tissue surface while minimizing undesirable heating below the surface of the tissue.

Another advantage of the present invention is provide a method for delivering energy to articular cartilage and particularly fibrillated articular cartilage, for treatment thereof, while minimizing collateral thermal effect on non-targeted portions and/or depths of the cartilage.

A further advantage of the present invention is to provide a method that can be practiced with a temperature controlled electrosurgical probe for minimizing and controlling chondrocyte death and improving safety.

Another advantage of the present invention is to provide a method of the above character in which sufficient thermal energy can be delivered to coagulate cartilage fibrils in predictable and reproducible levels thereby minimizing collateral damage when using a temperature-controlled device.

Yet another advantage of the present invention is to provide a method of the above character which can be used for treating chondromalacia and other articular cartilage defects.

The method for treating tissue of the present invention has other features and advantages which will be apparent from or are set forth in more detail in the accompanying drawings, which are incorporated in and form a part of this specification, and the following Detailed Description of the Invention, which together serve to explain the principles of the present invention.

### BRIEF DESCRIPTION OF THE DRAWINGS

FIG. 1 is schematic view of a system incorporating an apparatus for treatment of fibrillated tissue in use on a knee of a human body.

FIG. 2 is an enlarged schematic view of a knee capsule being treated by the system shown in FIG. 1.

FIG. 3 is an enlarged perspective view of an end of the apparatus shown in FIG. 1 treating a section of fibrillated tissue.

FIG. 4 is a graphic illustrating scanning electron microscopy (SEM) scores of a monopolar radio frequency energy (mRFE) treated surface at different lavage temperature/treatment time combinations.

FIG. 5 is an enlarged perspective view of an end of another apparatus which can be incorporated in the system of FIG. 1 for treatment of fibrillated tissue in use on a knee of a human body.

FIG. 6 is a cross-section view of the apparatus of FIG. 5 taken along line 6-6 of FIG. 5.

### DESCRIPTION OF THE PREFERRED EMBODIMENTS

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims.

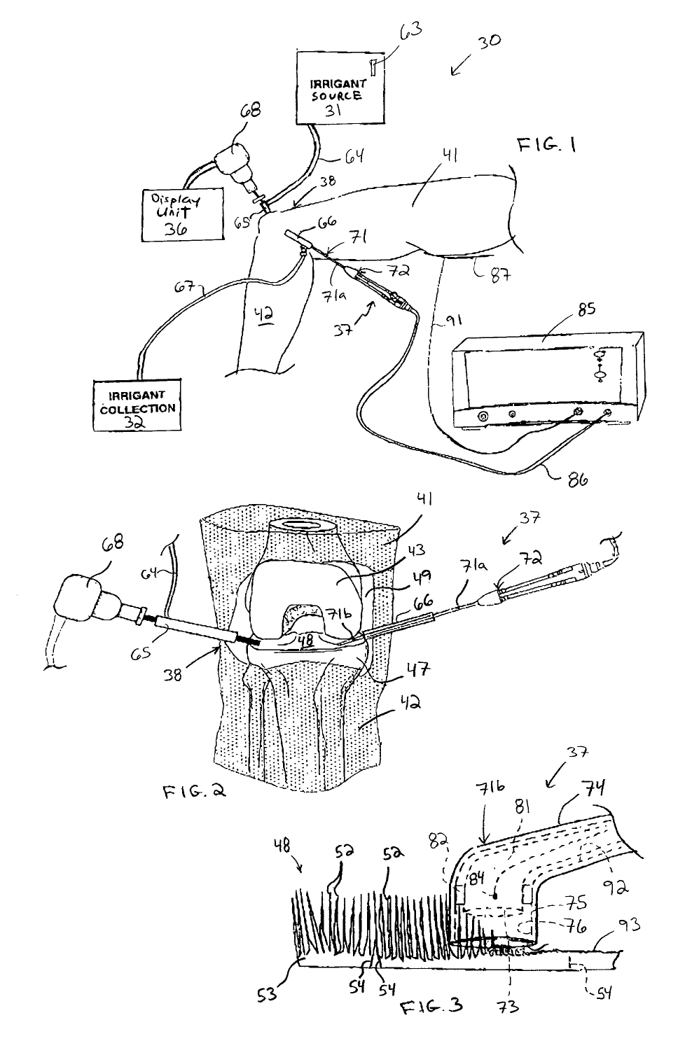

Turning now to the drawings, wherein like components are designated by like reference numerals throughout the various figures, attention is directed to FIGS. 1 and 2 which illustrate a system 30 with which the method for treating tissue in an arthroscopic environment of a mammalian body utilizing normothermic irrigating solution, that is, irrigating solution warmed to the normal body temperature of the mammalian body, can be performed in accordance with the present invention.

System 30 incorporates an irrigant source 31, an irrigant collection 32, a cathode ray tube or video display unit 36, and an apparatus 37 for treating a joint of a mammalian body. An exemplary knee joint 38 connecting a thigh 41 and a shin 42 is shown in FIGS. 1 and 2. Knee joint 38 is the junction of three bones, namely a thigh bone or femur 43, a shin bone or tibia 47, and a kneecap or patella (not shown). The ends of femur 43, tibia 47, and the patella are covered with articular cartilage 48 and are located within a joint capsule 49.

As shown in FIG. 3, cartilage or cartilage fibrils 52 may extend from a respective cartilage bed 53 for a length of approximately one to ten millimeters and often extend approximately four to seven millimeters. Disrupted articular cartilage 48 can further include fissures 54 and fragmented, avulsed or frayed cartilage. Hence, for purposes of the disclosure, disrupted articular cartilage 48 is broad enough to include cartilage that is fibrillated, fragmented and/or fissured.

The method of the present invention can be performed using the disclosed apparatus in combination with other standard arthroscopic implements such as an irrigating system, a viewing system and a positioning system in addition to the otherwise conventional equipment utilized in a minimally invasive procedure conducted on a mammal under general anesthesia. For example, a standard arthroscopic system such as the ones described in U.S. Pat. No. 6,068,628, the entire contents of which are incorporated herein by this reference, can be utilized for access to the joint capsule. Similarly, another arthroscopic system which can be utilized for access to the joint capsule is described in U.S. patent application Se. No. ______ [Attorney Docket No. A-69458/ENB/VEJ], filed Feb. 8, 2001 and entitled Method and Apparatus for Treatment of Disrupted Articular Cartilage, the entire contents of which are incorporated herein by this reference.

Turning now to the irrigating system, any suitable irrigant source can be utilized, such as solution bags (not shown) of normal or isotonic saline. In accordance with the present invention, the irrigant source provides normothermic irrigating solution, that is, solution which has been warmed to a temperature approximating the body temperature of the mammalian body upon which the method of the present invention is performed. In one embodiment, irrigating solution which is warmed to approximately 37° C., the body temperature of a human, is provided as a lavage for joint capsule 49. One should appreciate that the normothermic temperature may vary depending upon what type of mammal the method of the present invention is performed. The solution can be warmed to body temperature by placing bags of solution in a tissue bath, by a heat/stir plate device, or other means known in the art. In order to monitor temperature of the solution, a strip thermometer 63, which thermometer reads a different color depending upon the temperature sensed, or other well known means can be mounted on the bags of solution and/or solution source 31. For example, to ensure that irrigating solution is at the proper temperature, a bag of solution can be placed into a tissue bath for approximately 45 minutes to approximately one hour and/or until the thermometer indicates that the solution is the proper temperature.

An irrigating connection tube 64 includes tubing clamps or other suitable means for mechanically inhibiting and controlling the flow of the irrigating solution. A first percutaneous cannula 65 provides a portal for introducing irrigant into joint capsule 49 adjacent articular cartilage 48, as illustrated in FIGS. 1 and 2. A second cannula 66 provides a second portal or outflow port allowing irrigating fluid to exit joint capsule 49. Cannula 66 optionally includes a diversion tube 67 to direct the outflow of the irrigant away from an operator. One should appreciate that the irrigating system optionally may include a pump system that senses intra-articular pressure and maintains a desired pressure within joint capsule 49 to insure distension of the joint and adequate hemostasis. Alternatively, intra-articular pressure can be generated in a well known manner by elevating the solution bags above the level of the patient making use of a simple gravity supply.

Either one or both of cannulas 65 and 66 may be incorporated into a cannula system allowing the introduction of an arthroscopic scope 68 for viewing the interior of joint capsule 49 and distal extremity 71 b of probe member 71, as well as other interventional tools including other probes, cutting tools, electrosurgical instruments and electrothermal instruments which may be introduced into joint capsule 49. Arthroscopic scope 68 generally includes an optical rod lens which optionally is operably connected to a video camera that provides a video signal to a suitable display unit 36, such as a cathode ray tube, a liquid crystal display or a plasma monitor, for viewing by the operator.

Referring to FIGS. 1 and 2, apparatus 37 generally includes an elongate probe member 71 having a proximal extremity 71a and a distal extremity 71b. A probe handle 72 is mounted to proximal extremity 71a and an active electrode 73 (shown in FIG. 3) is provided on distal extremity 71b. One should appreciate that other probes can be used in accordance with the present invention. For example, other probes which can be utilized are described in U.S. patent application Ser. No. ______ [Attorney Docket No. A-69458/ENB/VEJ], filed Feb. 8, 2001 and entitled Method and Apparatus for Treatment of Disrupted Articular Cartilage, the entire contents of which are incorporated herein by this reference.

In one embodiment, probe member 71 includes an elongated and hollow outer shaft 74, as shown in FIG. 3. A peripheral wall 75 is formed by a distal extremity of outer shaft 74. Peripheral wall 75 defines a cavity 76. A lower edge of peripheral wall 75 defines a distal opening 80 communicating with cavity 76. Although the illustrated peripheral wall 75 is tubular, one should appreciate that it may take other forms. For example, the peripheral wall may be oval or polygonal in shape.

Active electrode 73 is made from any suitable conductive material such as stainless steel, platinum, iridium, titanium, silver and their alloys or any other medical grade metal. In the embodiment shown in FIG. 3, the electrode 73 has an outer surface having a convex and an outwardly bowed shape. It should be appreciated, however, that the outer surface of active electrode 73 can be planar, convex, or of any other suitable shape and be within the scope of the present invention.

Distal extremity 71b of probe member 71 includes an inner shaft 81 which is affixed to outer shaft 74 by a one or more brackets or spacers 82, as shown in FIG. 3. Conductive lead means is included with inner shaft 81 for providing energy to active electrode 73. Such conductive lead means can be in the form of a tubular member or tube, for example, inner shaft 81. Such conductive lead means can be made from any suitable conductive material and preferably a suitable medical grade conductor such as stainless steel 304 or any other stainless steel, MP35N, alloy metals, noble metals, any other suitable conductive carbon material or imbedded plastics or polymers. Active electrode 73 is secured to the distal end of inner shaft 81 by any suitable means so as to be electrically coupled to the active electrode. An additional tubular member or outer side wall, preferably in the form of a sleeve, is shrunk about or otherwise suitably disposed around the outside of inner shaft 81 and the side wall of the active electrode 73. Such a sleeve is preferably formed from a thermally-insulating material and is more preferably formed from TEFLON® (PTFE), polyolefin or nylon (PFA) or other plastics or polymers, serves to thermally insulate the side wall of active electrode 73 and electrode conductive inner shaft 81.

Spacers 82 are circumferentially disposed about the inner shaft 81 and serve to space active electrode 73 and the inner shaft 81 radially within outer shaft 74. The spacers 82 can be made from any suitable material such as glass, ceramic or any nonconductive electrical and/or thermal material. In one embodiment, active electrode 73 is spaced inwardly or proximally from opening 80 a distance of approximately two to ten millimeters and preferably approximately two to five millimeters so as to be recessed within distal extremity 71b. One should appreciate, however, that the method of the present invention may be performed using other types of probes, including probes having an active electrode that is not spaced from the opening.

A temperature or heat sensor 84 is preferentially carried by distal extremity 71b for measuring and monitoring the temperature of active electrode 73. Heat sensor 84 is of a conventional design and may consist of a thermocouple, a thermistor, a resistive wire, an integrated circuit (IC) or any other suitable sensor. The sensor 84 is electrically coupled to active electrode 73. Although sensor 84 of the illustrated embodiment is located within inner shaft 81 adjacent active electrode 73, one should appreciate that the heat sensor can be provided elsewhere provided that the heat sensor is capable of monitoring ambient temperature in the vicinity of the active electrode..

System 30 of the present invention is an electrothermal system which includes probe apparatus 37 and an energy source 85 to thermally coagulate disrupted articular cartilage, for example a fibrillated articular surface typically present in Grades I, II and III chondromalacia. Energy source 85 is preferably a radiofrequency (RF) generator and controller hereinafter referred to as RF generator 85. RF generator 85 includes a feedback controller which is dependent upon temperature and/or impedance. Active electrode 73 is electrically connected to RF generator 85 by means of conductive inner shaft 81 and a suitable connecting cable 86, which extends from the energy source 85 to probe handle 72 in order to electrically couple to the proximal end of inner shaft 81. As shown in FIG. 1, connecting cable 86 may be integrated to the probe handle 72 to form a one-piece unit between apparatus 37 and probe handle 72. This provides a fluid resistant environment within electrosurgical probe handle 72 to prevent electrical disconnects and shorting between apparatus 37 and energy source 85. It will also be appreciated that probe handle 72 and connecting cable 86 may also be separate units utilizing a keyed and/or electrically insulated connection at a proximal end of probe handle 72.

In one embodiment, a grounding pad 87 is provided on thigh 41 of a patient's body as shown in FIG. 1. The grounding pad 87 may also be placed on any electrically suitable location of the body to complete the circuit. Grounding pad 87 is electrically connected to radio frequency generator 85 via a second return connecting cable 91 to complete the electrical circuit. RF generator 85 can deliver high frequency or radiofrequency voltage in the range of one to 350 watts.

Optionally, impedance is monitored by energy source 85 along the electrical circuit between power output and return input of the energy source 85. The energy source 85 monitors the impedance of the electrical circuit by measuring the difference between the output power and the input return as a function of voltage over current. In a typical monopolar system the impedance level is about 100 ohms and in a typical bipolar system the impedance level is about 60 ohms.

The feedback controller of RF generator 85 monitors the temperature of the tissue or cartilage being treated by monitoring the temperature experienced by sensor 84 located in the proximity of active electrode 73. The feedback controller compares such temperature to a programmed temperature profile. The feedback control can also directly monitor system impedance of the electrical circuit. If the measured impedance exceeds a predetermined level, energy delivery to active electrode 73 is disabled or adjusted thus ceasing or adjusting delivery of thermal energy to active electrode 73. If the temperature within cavity 76 measured by sensor 84 exceeds a predetermined desired temperature, energy delivery to active electrode 73 is disabled or adjusted thus ceasing or adjusting delivery of thermal energy to active electrode 73.

Optionally, apparatus 37 may be used in combination with a suction source. For example, the probe member includes a lumen 92, as shown in FIG. 3, which extends from cavity 76 towards proximal extremity 71a (not shown in FIG. 3) of the probe member and through probe handle 72. In the illustrated embodiment, lumen 92 is annular in cross section at distal extremity 71b where the lumen communicates with cavity 76. Specifically, such annular lumen 92 is formed at its outside by peripheral wall 75 and at its inside by inner shaft 81. Lumen 92 fluidly connects with the suction source via a suitable fluid coupling adjacent proximal extremity 71a in a conventional manner. In such configuration, the suction source can be activated to produce a suction effect within lumen 92 and cavity 76. The suction source can be activated by a physician to aspirate the joint cavity as desired by the physician. When the suction source is activated, fluid, particulates and other matter within the surgical field is aspirated into a collection vessel, for example, irrigant collection 32. One should appreciate, however, that apparatus 37 may be used with or without a suction source.

In operation and use, a suitable positioning system can be used to immobilize joint 38 to facilitate the operator's or physician's access to joint capsule 49. The positioning system is selected based upon the specific anatomy to be addressed with the procedure in accordance with the present invention.

After the patient has been appropriately sedated or anesthetized, joint capsule 49 is pressurized by a suitable irrigant to create a work area within the joint space 49, as shown in FIG. 2. For example, fluid inflow from irrigant source 31 by means of pump and/or gravity introduces pressurized irrigant fluid into joint capsule 49 so as to create a workspace within joint capsule 49 as well as to provide a flushing and a warming, temperature stabilizing effect.

In contrast to prior methods in which the irrigating solutions are commonly stored in the operating room and are then used at room temperature, that is approximately 19°-22° C., the irrigating solution is pre-warmed to a temperature approximating the body temperature of the mammalian body upon which the method of the present invention is practiced. The saline or other irrigating fluid from irrigant source 31 further serves to stabilize the temperature of cartilage bed 53 and surrounding tissue within joint capsule 49.

Probe handle 72 is grasped by the physician to introduce distal extremity 71b of probe member 71 through cannula 66 and into the joint capsule of the patient and thereafter to position lower edge 56 of distal extremity 71b adjacent disrupted articular cartilage 48. Although distal extremity 71b is shown to be substantially flush against articular cartilage 48, one should appreciate that the actual placement of the probe member with respect to the articular cartilage will depend upon the actual probe member used. Scope 68 allows the physician to view distal extremity 71b within joint capsule 49 and thus facilitates movement of distal extremity 71b relative to articular cartilage bed 53 by the physician.

Probe member 71, namely distal extremity 71a, is swept across the surface of articular cartilage bed 53. The physician activates RF generator 85 and RFE is supplied to active electrode 73. The saline and/or other conductive irrigants present within joint capsule 49 serve to transmit such RFE and, together with other tissue of the mammalian body, transmit the RFE to grounding pad 87. The passing of such radio frequency through the fluid heats such fluid to a temperature that can be monitored by temperature sensor 84. The amount of energy supplied to electrode 73 controls the temperature of the electrode.

The disrupted articular cartilage which is immediately adjacent active electrode 73, for example, the fibrillated articular cartilage fibrils or strands 52 extending from cartilage bed 53 over which cavity 76 rests, are thermally treated by the heated fluid within cavity 76 so as to become coagulated cartilage. Fibrillated strands 52 which contact distal surface 38 of active electrode 73 are similarly coagulated or melded and thus treated. Subjecting the fibrillated articular cartilage strands 52 to temperatures in the range of approximately 45° C. to 100° C., preferably in the range of approximately 45° C. to 85° C., and more preferably in the range of approximately 45° C. to 60° C., causes the fibrillated articular cartilage strands 52 to meld into cartilage bed 53 and thus form a substantially smooth coagulated mass on the surface of the cartilage bed 53 as indicated by numeral 93 in FIG. 3. In this manner, the cartilage bed 53 is sealed into a coagulated mass 93. The treatment of disrupted articular cartilage 48 by apparatus 37 in the foregoing manner can also result in the sealing of fissures 54, one of such sealed fissures 54 being shown by a dashed line in FIG. 3, and the sealing of any fragmented, avulsed or otherwise disrupted cartilage into a coagulated mass 93.

In the illustrated embodiment, active electrode 73 is spaced or recessed inwardly from opening 80 so as to minimize direct contact between the active electrode and cartilage bed 53 when apparatus 37 is utilized for treating fibrillated articular cartilage strands 52. Active electrode 73 is recessed within opening 80 a distance that allows for the targeted fibrillated articular cartilage strands 52 to extend into the cavity or space created by the extension of peripheral wall 75 beyond distal surface 38 of the active electrode. The distance between the active electrode and the surface of the articular cartilage bed 53 is preferably such that the delivery of energy from RF generator 85 coagulates the fibrillated articular cartilage strands into a coalesced and singular mass to form a contiguous articular cartilage surface. Such distance reduces the delivery of thermal energy to underlying subchondral bone thus preventing avascular necrosis (AVN). The movement of apparatus 37 by the operating physician across the disrupted articular cartilage 48 limits the time of exposure of such cartilage to thermal heating, which is also a factor in preventing AVN. As noted above, the active electrode need not be spaced from opening to fall within the scope of the present invention.

As thermal energy is so delivered to active electrode 73, the physician advances or sweeps probe member 71 continuously across cartilage bed 53 at a speed that allows for sufficient coagulation of fibrillated articular cartilage strands 52 to occur and form a coagulated mass 93, as shown in FIG. 3, but without excessive thermal exposure to deeper viable tissues including cartilage bed 53 and subchondral bone such as tibia 47 (FIG. 2). The sweeping motion of the probe member along cartilage bed 53 results in a convective thermal effect that follows the path of the probe.

One should appreciate that tissues do not immediately heat up when exposed to thermal energy. The exposure time of thermal energy upon an area of cartilage bed 53 is a factor in treatment effectiveness. The phenomena known as thermal latency of tissues determines the thermal response time, or thermal conduction time of the targeted tissue being treated. In accordance with the present invention, the use of normothermic irrigating solution reduces the effects of thermal latency because the temperature differential is reduced. In particular, the temperature differential between ambient temperature and treatment temperature when normothermic irrigating solution is used is less then the temperature differential when room temperature or precooled irrigating solutions are used.

Temperature sensor 84 permits the ambient temperature of joint capsule 49 to be accurately monitored. Accordingly, the temperature of electrode 73 can be accurately monitored and regulated thereby minimizing the possibility of thermal damage to non-targeted tissue as well as to apparatus 37. For example, because the temperature is accurately monitored, predictable and reproducible levels of energy can be delivered in order to effectively meld fibrillated articular cartilage strands 52 and minimize collateral thermal effect on non-targeted tissue including underlying cartilage bed 53 and subchondral bone 47.

Advantageously, using a warmed irrigating solution, for example, a warmed lavage having a temperature approximating the body temperature of the mammalian body to be treated can significantly decrease the depth of chondrocyte death. As noted above, warmed irrigating solution serves to stabilize the temperature of cartilage bed 53 and surrounding tissue within joint capsule 49. Such temperature stabilization advantageously minimizes the thermal heating of the deeper layers of cartilage bed 53 and thus inhibits the undesirable thermal damage of such deeper tissues, as is demonstrated in the following exemplary study. The study determined that normothermic lavage solution, that is, lavage solution warmed to the normal body temperature of the body to the treated, limits the depth of chondrocyte death when a temperature controlled monopolar RFE (mRFE) treatment was used to perform thermal chondroplasty as compared to room temperature lavage solution, that is, approximately 22° C. In the case of a treating a human body, the normothermia lavage solution is warmed to approximately 37° C.

In the exemplary study, sixteen fresh osteochondral sections from sixteen patients undergoing total or partial knee arthroplasty were used to complete the study. Chondromalacia was graded using a modified Outerbridge system in which softened cartilage surface is designated as “Grade 1”, softened cartilage with fine fibrillations as “Grade 2”, fibrillated surface with pitting to subchondral bone as “Grade 3”, and fibrillation of cartilage and exposed subchondral bone as “Grade 4”. To avoid experimental bias, each graded osteochondral section was cut into two sections. One section was treated with monopolar radiofrequency energy (mRFE) in physiologic saline (0.15M) at 22° C. (room temperature) whereas another section was treated in physiologic saline (0.15M) at 37° C. An area 2 cm distant from the radiofrequency energy (RFE) treated area on each specimen served as control.

For 37° C. lavage solution, 1 liter of physiologic saline was placed in a plastic container heated by a NUOVA II hot plate and stirrer (Thermolyne Corporation, Dubuque, Iowa, USA). A thermometer was used to monitor the temperature, and the saline was maintained at 37° C. for 60 minutes. After temperature stabilization, cartilage sections were placed in the saline and allowed to equilibrate for 20 minutes so that sections reached 37° C. prior to mRFE treatment. No fluid flow was used during mRFE treatment based on the results from a previous study that determined the negative effect of irrigation fluid flow on cartilage matrix temperatures during mRFE chondroplasty. A Vulcan EAS™ coupled with a TAC-C II probe (Oratec Interventions, Inc, Menlo Park, Calif.) was used to deliver mRFE in a light contact fashion over a 1.0-cm² area on each section in a paintbrush treatment pattern at a generator setting of 70° C. and 15 watts. RFE treatment times of 10 sec and 15 sec were evaluated in the study. For each treatment time/lavage temperature combination, eight sections were tested (total, 32 treatments, 4 groups, n=8). Ten and 15 second treatment times were selected.

After RFE treatment, each treated area was processed for analysis by vital cell staining/confocal laser microscopy (CLM) and scanning electron microscopy (SEM). A diamond-wafering blade (ISOMET® 2000 Precision Saw; Buehler LTD. Corporation, Lake Bluff, Ill., U.S.A.) was used to cut 1.5-mm thick osteochondral sections for CLM. Phosphate buffered saline (PBS) was used for irrigation to avoid thermal injury during sectioning as previously described. Sections were placed in 1.0-ml PBS and maintained at 4° C. for 3 hours prior to staining for cell viability.

Cell viability staining was performed using ethidium homodimer (EthD-1) and (acetoxymethylester) calcein-AM in conjunction with CLM. The 1.5-mm sections were stained by incubation in 1.0-ml of PBS containing 1.0-mL calcein-AM per 10-mL EthD-1 (LIVE/DEAD® Viability/Cytotoxicity Kit (L-3224), Molecular Probes, Eugene, Oreg.) for 30 minutes at room temperature. The 1.5-mm osteochondral section was placed on a glass slide, moistened with several drops of PBS. A confocal laser microscope (BIO-RAD® MRC-1000, Bio-Rad, Hemel Hampstead/Cambridge, England) equipped with an argon laser and necessary filter systems (fluorescein and rhodamine) was used with a triple labeling technique. In this technique, the signals emitted from the double-stained specimens can be distinguished because of their different absorption and emission spectra These images are displayed on a monitor in a RGB (red, green and blue) mode. All cartilage samples were coded so that treatment time and lavage solution temperature were unknown to the examiners.

The depth of chondrocyte death of each section was determined for each RFE treated region in the CLM image, and all images coded to prevent identification of the lavage temperature and treatment time applied. The CLM was calibrated using a micrometer measured through the objective lens (2×) used for this project (20×total magnification; objective +eyepiece magnification). The pixel length measured on images was converted to micrometers as previously described. The depth of chondrocyte death was determined for each confocal image of the osteochondral sections with Adobe PhotoShop™ (Adobe PhotoShop, Version 5.5, San Jose, Calif.).

After evaluation by CLM, the same cartilage specimens were trimmed (4×3×1.5 mm) and fixed in modified Kamovsky's solution (2% glutaraldehyde in 0.1 mol/L sodium cacodylate buffer, pH 7.4) for 2 hours and then washed in 0.1 mol/L sodium cacodylate buffer twice at room temperature. These samples were stored in 0.1 mol/L sodium PBS for 8 hours at 4° C. After dehydration in a graded series of ethanol (50%, 70%, 80%, and 100%) and air drying, the samples were coated with gold in a Bio-Rad E5000M gold coater and examined with a Hitachi S570 scanning electron microscope. The image of each section was coded so that the lavage temperature and treatment time were unknown. The SEM images were scored by three investigators independently with a custom designed scoring system as previous study described. Higher scores indicate a smoother cartilage surface.

Mean depth of chondrocyte death, mean mRFE delivery power, time to reach RFE preset temperature, and mean mRFE treatment temperature (temperature measured from thermocouple located within the RF probe tip) were compared among groups of lavage temperatures and treatment time combinations using ANOVA (SAS version 7.1, SAS Institute, Cary, N.C., USA). Factors included in the analysis were patient, treatment time, and lavage solution temperature. When differences among groups were demonstrated by ANOVA, appropriate post hoc tests were employed. Paired t-tests were used to compare the effect of lavage solution temperature within treatment time groups. Patient gender was compared using Wilcoxon sign rank tests. The inter- and intra-observer precision errors were determined for the SEM scores. The Kruskal-Wallis test was used to compare the SEM image scores between different lavage temperatures at the same treatment time. When significance was identified using the Kruskal-Wallis test, the Mann-Whitney procedure was used to compare the subjective scores between groups. P-values less than 0.05 were considered significant.

The results of the above study indicated that there were no significant differences in age or gender among treatment groups (mean age, 65±7 years; 7 males and 9 females; p>0.05).

CLM demonstrated that the depth of chondrocyte death in 37° C. lavage solution was significantly less than that in 22° C. solution at both 10 and 15 sec treatment times (FIG. 1, Table 1). SEM demonstrated that cartilage surfaces were smoothed in both 37° C. and 22° C. lavage solutions treated for both 10 sec and 15 sec treatment times compared with the control specimens.

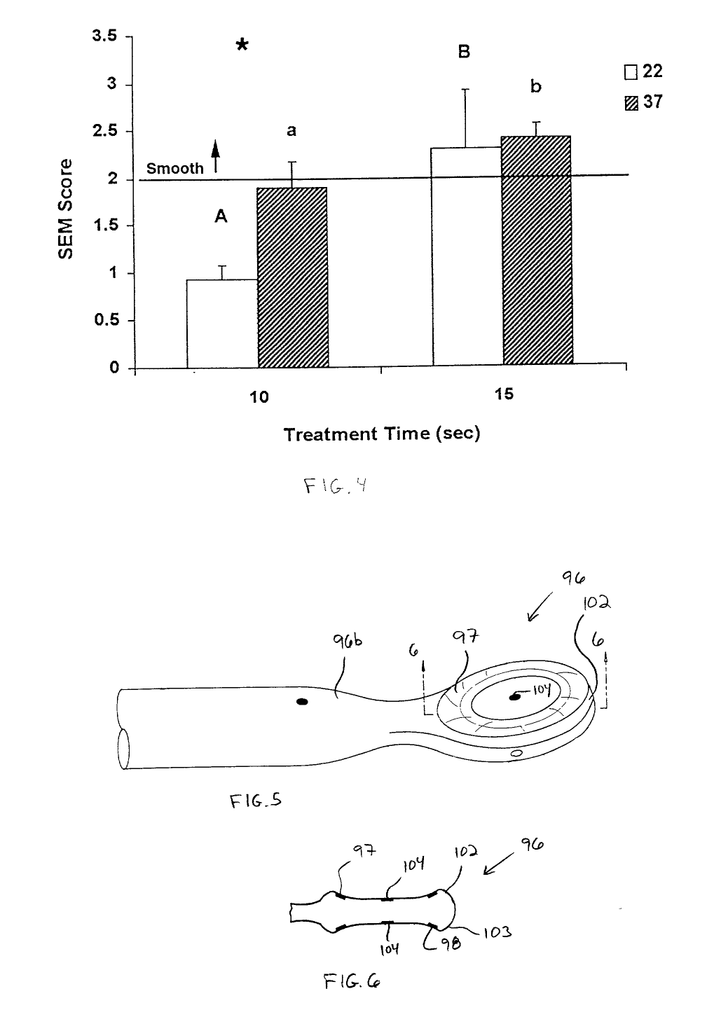

SEM demonstrated that chondromalacic cartilage surfaces treated by RFE in 37° C. lavage solution were smoother than those treated in 22° C. solution for 10 sec (p<0.05), but that there were no differences in surface smoothing between sections treated in 37° C. and 22° C. lavage solution for 15 sec treatment, as shown in FIG. 4. Higher scores indicate smoother surface, as indicated by the vertical arrow. Score values represent the means of three observers±standard deviation. Means with different letters are significantly different from each other at different RFE treatment time intervals (p<0.05). Means with asterisk are significantly different from each other at different lavage temperatures (p<0.05). Scores above transverse line at score 2 mean that cartilage surfaces were smoothed.

As shown in FIG. 4, chondromalacic cartilage surfaces treated by RFE for 15 sec were smoother than those treated for 10 sec treatment time group in both 37° C. and 22° C. lavage solutions (p<0.05). The intra- and inter-observer precision errors for SEM scores were 10.8% and 12.9% respectively.

The mean mRFE treatment temperatures in 37° C. lavage solution was higher than in 22° C. lavage solution for both 10 sec and 15 sec treatment times (p<0.05), whereas RFE delivery power in 37° C. was lower than 22° C. lavage solution for both treatment times (p<0.05).

TABLE 1
The Effects of Lavage Temperature^{Ψ}

| Treatment | | | | |
|---|---|---|---|---|
| Time | 10 sec | | 15 sec | |
| Lavage temp | | | | |
| (° C.) | 22 | 37 | 22 | 37 |
| Depth of | 620 ± 106 | 420* ± 219 | 930 ± 236 | 590* ± 214 |
| chondrocyte | | | | |
| death (μm) | | | | |
| Mean power | 8.5 ± 0.6 | 5.9* ± 0.9 | 7.6 ± 0.8 | 5.1* ± 0.4 |
| (watts) | | | | |
| Time to set | 1.8 ± 0.5 | 0.7* ± 0.2 | 1.3 ± 0.5 | 1.0* ± 0.5 |
| temp (sec) | | | | |
| Mean probe | 67.5 ± 1.1 | 70.1* ± 0.8 | 68.7 ± 0.6 | 70.3* ± 0.4 |
| temp (° C.) | | | | |

*Indicates significant difference between lavage temperatures at each treatment time (p < 0.05).
^{Ω}mean ± S.D.

The times for mRFE to reach preset temperature were faster in 37° C. lavage solution than in 22° C. lavage solution for both 10 sec and 15 sec mRFE treatment times (p<0.05). mRFE treatment temperatures were more stable in 37° C. lavage solution (coefficient of variation (CV)=6.23%) compared to 22° C. lavage solution (CV=7.92%) (p<0.05).

Thermal chondroplasty performed with mRFE in 37° C. lavage solution caused significantly less chondrocyte death than in 22° C. lavage solution. Increasing the lavage solution temperature allowed the probe tip to reach preset temperature more rapidly and resulted in less total power (energy) delivery while still effectively smoothing the cartilaginous surface.

The goal of this study was to determine if normothermic lavage solution (37° C.) would limit the depth of chondrocyte death when temperature controlled mRFE was used to perform thermal chondroplasty compared to room temperature lavage solution (22° C.). This hypothesis was based on the mRFE's design and temperature control algorithm. The mRFE system evaluated (Vulcan EAS™ RF generator, Oratec Interventions, Inc, Menlo Park, Calif.) uses delivered power to control the tissue temperature reflected by a thermocouple within the mRFE probe tip. At the beginning of treatment, the RF generator delivers full preset power to cause tissue heating. The thermocouple within the mRFE probe tip is subsequently heated, reaching the preset temperature relatively quickly. After reaching the preset temperature, the mRFE algorithm reduces the power to decrease tissue/probe-tip temperature and then uses minimum power output to maintain the tissue temperature near the preset temperature. This results in the mRFE generator delivering mean powers that are significantly less than preset powers (34-57% of preset power in this study) to maintain the preset temperatures.

The results of this study demonstrated that thermal chondroplasty performed with mRFE in 37° C. lavage solution caused significantly less chondrocyte death than that in 22° C. lavage solution. The explanation for this decreased cell death is that less delivered power (energy) resulted in less chondrocyte injury. The delivered power in 37° C. lavage solution was approximately 40% less than that in 22° C. lavage solution during both 10 and 15 sec treatment times. Delivered power equals the electric current multiplied by electric voltage. Organ et al. reported that RFE current intensity (I) had a very strong influence on the lesion generated. The lesion size increased as I². The temperature controlled mRFE device tested in this study was able to maintain the probe tip temperatures equivalent to preset temperature at lower mean powers in 37° C. compared to 22° C. lavage solution.

In addition, the results of this study demonstrated that the time to reach preset temperature at the initiation of treatment in 37° C. lavage solution was significantly faster than in 22° C. lavage solution in both 10 and 15 sec treatment groups. This likely occurred because the temperature difference between the lavage solution and RFE preset temperature was 33° C. for the 37° C. group and 48° C. for the 22° C. group.

In this study, SEM demonstrated that there were no significant differences in cartilage surface smoothing and contouring between the 37° C. lavage solution and the 22° C. lavage solution for the 15 sec treatment time group. However, mRFE treatment of the cartilage surface in 37° C. lavage solution for 10 sec resulted in a significantly smoother surface than the same treatment time in 22° C. lavage solution. This probably was caused by the faster time to preset temperature in the 37° C. lavage solution group (0.7 vs 1.8 sec) and the higher mean temperature reached with 37° C. group (70.1° C. vs 67.5° C.). The major reason why mean mRFE treatment temperature in 37° C. lavage solution was significantly higher than that in 22° C. lavage solution during mRFE treatment was that the temperature fluctuation in 37° C. lavage solution was less than in 22° C. lavage solution. The mRFE generator is able to maintain probe tip temperature closer to the preset temperature in 37° C. lavage solution than in 22° C. lavage solution, with lower delivered power.

Advantageously, this ex vivo study indicated that thermal chondroplasty with mRFE using 37° C. lavage solution significantly reduced chondrocyte death compared to using the standard room temperature (22° C.) lavage solution. During 10 and 15 sec treatment times over a 1 cm² area of grade 2 chondromalacic cartilage, the mean depth of chondrocyte death ranged from 420-590 μm. This depth is similar to expected depth of chondrocyte loss produced by mechanical debridement and shaving. Compared to mechanical debridement with a shaver, mRFE has several advantages: 1) a smoother surface may be produced, 2) injury to adjacent and untreated regions may be more easily avoided, and 3) rapid and easy contouring is achieved that may result in shortened operative process.

In addition to the above advantages, the method for treating tissue using normothermic lavage in accordance with the present invention minimizes undesirable heating below the surface of the tissue thereby resulting in less depth of chondrocyte death and produces smoother surfaces as compared to other methods using cooler lavages. Advantageously, the method of the present invention requires less energy to heat tissue, including articular cartilage to be treated, and requires less power to maintain probe temperature. As less power is required to maintain probe temperature, thermal energy can be delivered to the probe in predictable and reproducible levels in such a manner that the feedback controller is less likely to overcompensate in maintaining probe temperature.

The structure of the apparatus and probe member with which the present method is utilized may vary widely and fall within the scope of the present invention. For example, the probe members may have a variety of different geometric configurations. For example, the electrode may be spherical, flat, asymmetric or concave. In addition, it should be appreciated that the energy source, apparatus and method of the present invention can utilize other suitable frequencies along the electromagnetic spectrum, including infrared, coherent light, sonic and microwave, for heating the disrupted articular cartilage exposed thereto and be within the scope of the present invention.

In another embodiment, and elongate probe member 96 as shown in FIGS. 5 and 6 is utilized instead of elongate probe member 71. Elongate probe member 96 is similar to that shown in U.S. Pat. No. 6,068,628, the entire content of which is incorporated by this reference. A distal extremity 96b of elongate probe member 96 includes first and second annular electrodes 97 and 98 which are formed on a periphery of surfaces 102 and 103, respectively. A temperature sensor 104, similar to heat sensor 84 discussed above, is provided on the distal extremity of probe member 96 to monitor ambient temperature adjacent the electrodes.

Probe member 96 can be operated in either a monopolar or a bipolar mode. In particular, probe member 96 can be operated in a bipolar mode as it includes an active electrode 97 and a return electrode 98 provided on an external surface surfaces 102 and 103. Similar to probe member 71, active electrode 97 is electrically connected to the RF generator 85. Return electrode 98 is also electrically connected to the RF generator and competes the electrical circuit therewith instead of a grounding pad. The bipolar current path extends from active electrode 97 to return electrode 98 in a well known manner.

In use and operation, probe member 96 is used in substantially the same manner as probe member 71 to apply thermal energy to tissue in an arthroscopic environment. Similarly, the method of the present invention utilizing warmed irrigating solution can be practiced with probe member 96 in substantially the same manner as probe member 71 discussed above. One should appreciate that the method of the present invention can similarly be practiced using a wide variety of probe members designed and configured to apply thermal energy to a tissue in an arthroscopic environment.

One method for treating tissue having a surface in an arthroscopic environment of a mammalian body having a body temperature with a probe having a proximal end and an electrode at a distal end in accordance with the present invention includes the steps of providing a warmed irrigating solution having a temperature approximating the body temperature, delivering the warmed irrigating solution into the arthroscopic environment, introducing the distal extremity of the probe into the arthroscopic environment, positioning the electrode adjacent the surface of the tissue and supplying thermal energy to the electrode so as to treat the tissue. The warmed irrigating solution inhibits undesirable heating below the surface of the tissue.

The warmed irrigating solution may be selected from the group consisting of normal saline, ringers lactated solution, Glycine and bacteriostatic water. The warmed irrigating solution may have a temperature of approximately 37° C. and may be warmed by a tissue bath.

The method may further include the step of monitoring the ambient temperature within the arthroscopic environment with a sensor carried by the distal extremity of the probe.

The monitoring step may further include the step of modulating the amount of thermal energy supplied to the electrode in response to the ambient temperature within the arthroscopic environment.

The supplying step may further include the step of supplying radio frequency energy to the electrode. The supplying step may further include the step of supplying radio frequency energy between the electrode and a return electrode, the electrode and the return electrode being coupled to a radio frequency generator. The return electrode may be carried by the distal extremity of the probe.

The surface may be a fibrillated cartilage surface, in which case, the supplying step includes the step of supplying sufficient thermal energy to the electrode to reduce the level of fibrillation at the fibrillated cartilage surface.

Another method for treating tissue having a surface in an arthroscopic environment of a mammalian body having a body temperature with a probe having a proximal end and an electrode at a distal end in accordance with the present invention includes the steps of providing a warmed irrigating solution having a temperature approximating the body temperature, delivering the warmed irrigating solution into the arthroscopic environment, introducing the distal extremity of the probe into the arthroscopic environment, positioning the electrode adjacent the surface of the tissue, supplying radio frequency energy to the electrode so as to treat the surface of the tissue whereby the warmed irrigating solution inhibits undesirable heating below the surface of the tissue and monitoring the temperature of the arthroscopic environment so as to modulate the supply of radio frequency energy to the electrode in response to such monitored temperature.

The supplying step may further include the step of coupling the electrode to a radio frequency generator. The supplying step may include the step of coupling a return electrode to the radio frequency generator so that the radio frequency energy passes between the electrode and the return electrode. The return electrode may be carried by the distal extremity of the probe.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A method for treating tissue having a surface in an arthroscopic environment of a mammalian body having a body temperature with a probe having a proximal end and an electrode at a distal end, comprising the steps of providing a warmed irrigating solution having a temperature approximating the body temperature, delivering the warmed irrigating solution into the arthroscopic environment, introducing the distal extremity of the probe into the arthroscopic environment, positioning the electrode adjacent the surface of the tissue and supplying thermal energy to the electrode so as to treat the tissue whereby the warmed irrigating solution inhibits undesirable heating below the surface of the tissue.

2. The method of claim 1 wherein the warmed irrigating solution is selected from the group consisting of normal saline, ringers lactated solution, Glycine and bacteriostatic water.

3. The method of claim 2 wherein the warmed irrigating solution has a temperature of approximately 37° C.

4. The method of claim 1 wherein the providing step includes the step of providing an irrigation solution warmed by a tissue bath.

5. The method of claim 1 further comprising the step of monitoring the ambient temperature within the arthroscopic environment.

6. The method of claim 5 wherein the monitoring step includes the step of monitoring the ambient temperature within the arthroscopic environment with a sensor carried by the distal extremity of the probe.

7. The method of claim 5 wherein the supplying step includes the step of modulating the amount of thermal energy supplied to the electrode in response to the ambient temperature within the arthroscopic environment.

8. The method of claim 1 wherein the supplying step includes the step of supplying radio frequency energy to the electrode.

9. The method claim 8 wherein the supplying step includes the step of supplying radio frequency energy between the electrode and a return electrode, the electrode and the return electrode being coupled to a radio frequency generator.

10. The method of claim 9 wherein the return electrode is carried by the distal extremity of the probe.

11. The method for claim 1 wherein the surface is a fibrillated cartilage surface, the supplying step includes the step of supplying sufficient thermal energy to the electrode to reduce the level of fibrillation at the fibrillated cartilage surface.

12. A method for treating tissue having a surface in an arthroscopic environment of a mammalian body having a body temperature with a probe having a proximal end and a radio frequency electrode, comprising the steps of providing a warmed irrigating solution having a temperature approximating the body temperature, delivering the warmed irrigating solution into the arthroscopic environment, introducing the distal extremity of the probe into the arthroscopic environment, positioning the electrode adjacent the surface of the tissue, supplying radio frequency energy to the electrode so as to treat the surface of the tissue whereby the warmed irrigating solution inhibits undesirable heating below the surface of the tissue and monitoring the temperature of the arthroscopic environment so as to modulate the supply of radio frequency energy to the electrode in response to such monitored temperature.

13. The method of claim 12 wherein the supplying step includes the step of coupling the electrode to a radio frequency generator.

14. The method of claim 13 wherein the supplying step includes the step of coupling a return electrode to the radio frequency generator so that the radio frequency energy passes between the electrode and the return electrode.

15. The method of claim 14 wherein the return electrode is carried by the distal extremity of the probe.
